(19) United States
(12) Patent Application Publication
(10) Pub. No.: US 2019/0151067 A1
(43) Pub. Date: May 23, 2019

(54) Arrangement for Storing and Loading a Self-Expanding Stent-Like Device

(71) Applicant: QVANTEQ AG, Zürich (CH)

(72) Inventors: Arik ZUCKER, Zürich (CH); Armin W. MÄDER, Richterswil (CH); Stefano BUZZI, Birmensdorf (CH)

(73) Assignee: Qvanteq AG, Zürich (CH)

(21) Appl. No.: 16/097,770

(22) PCT Filed: May 9, 2017
(86) PCT No.: PCT/EP2017/060998

§ 371 Date: Oct. 30, 2018

(30) Foreign Application Priority Data
May 9, 2016 (CH) 00605/16

(51) Int. Cl. A61F 2/00; A61F 2/01; A61F 2/966

(52) U.S. Cl. CPC A61F 2/0095; A61F 2002/91541; A61F 2/966; A61F 2/01

(57) ABSTRACT

An arrangement for storage of a self-expanding stent-like device includes a container for storing the stent-like device and a stent delivery system arranged at the container and including an outer tube and an inner shaft axially moveable relative to the outer tube. The container includes an interior space and one stent passage opening. A distal end of the outer tube is connected to the stent passage opening. An insertion part of ring-like shape is located at the stent passage opening. The inner shaft extends through the insertion part and includes a retainer to prevent the stent-like device from axial movement relative to the inner shaft. When the stent-like device is stored in the interior space of the container, a proximal end of the stent-like device is compressed to a diameter smaller than a diameter of the insertion part and is held radially compressed by the insertion part. The stent-like device is safely stored inside the container and can be prepared for implantation by continuous pulling of the inner shaft, which loads the stent-like device into the outer tube and detaches the delivery system from the container.

### CROSS-REFERENCE TO RELATED APPLICATIONS

This application is the U.S. National Phase of International Patent Application No. PCT/EP2017/060998, filed on 9 May 2017, which claims benefit of Swiss Patent Application No. 00605/16, filed on 9 May 2016, the contents of which are incorporated herein by reference in their entirety.

### BACKGROUND

### Field

The present invention relates to an arrangement for storage of a self-expanding stent-like device, in particular a self-expanding stent, including a container for storing the stent and a stent delivery system arranged at the container. Further, the arrangement serves for loading the stent or stent-like device from the container onto the delivery system for preparing the stent to be delivered into a body lumen.

### Related Art

Stents are used as a medical implant for treating lesions in a body lumen in many different medical applications. A distinction is substantially made between balloon-expanding and self-expanding stents. Self-expanding stents are well known in the prior art. They can consist of a metal, polymer or a combination thereof having memory effect and can be radially compressed against their elastic radial outward force in order to be inserted into a catheter delivery system and subsequently into a body lumen. They are released from the catheter at the application site, the impaired lesion, and due to their radial outward force they get back into their expanded state.

Medical practitioners want a rapid healing of a stent, the so-called reendothelialization, which means a coverage of the implanted stent by a layer of cells forming a tissue. Preferably, this tissue layer is very thin and smooth and covers the entire stent. This is of the utmost importance for the success of the stent therapy because the cells in the endothelial layer form essential antithrombotic factors. However, as long as the stent has not entirely healed, and the structures thereof are exposed to the blood flow, it is of the utmost importance to provide an antithrombotic stent surface. It is well-known that stents with hydrophilic surface properties have a much higher hemocompatibility, e.g., a much lower thrombogenicity. The hydrophilicity of a stent surface can be increased by substances applied onto the stent surface, for example by coating methods, or by a surface treatment of the stent surface such as polishing, grinding, or etching. On a stent surface showing a high hydrophilicity, molecular chemical impurities originating from the atmosphere, primarily hydrocarbon compounds, are significantly reduced on the surface by such treatment, whereby the contact angle of a water droplet present on the surface as a measure of the hydrophilicity is reduced with respect to the contact angle prior to such treatment. The hydrophilic stent surface is extremely susceptible to contamination and therefore requires specific packaging, which prevents re-contamination of the stent surface. For example, the stent can be stored in an inert environment, as known from WO 2010/000080.

In contrast, the main purpose of a delivery system is delivering the self-expanding stent or stent-like device to the application site in a body lumen, where the stent or stent-like device needs to be released for implantation. Since delivery and implantation of stents or stent-like devices can be a big challenge, the delivery systems are designed in terms of shape and materials for optimal delivery. However, some materials used for optimal delivery systems may not be suitable for an inert packaging of the stent or stent-like device. Many times at least a part of the delivery device protrudes into the packaging with the inert filling and gets in touch with the inert filling. It is important to keep in mind that the inert filling has an inert characteristic with respect to the quality of the stent surface, meaning that it hinders or minimizes recontamination of the stent surface, and therefore the surface can keep its hydrophilic property. The filling, on the other hand, might not be inert with respect to the materials used for the delivery system. And therefore, when such materials get in touch with the filling inside a packaging, the filling can act as a solvent into which molecules or even parts of the materials of the delivery system are eluted. Such elutions negatively impact the hydrophilic property of the stent surface.

Further, it is common to all stents or stent-like devices that they need to have a smaller diameter for being introduced into a body lumen than when they carry out their function in the body. In general, the manufacturer pre-mounts the stents or stent-like devices on a catheter and packages them. However, a stent or stent-like device may also be compressed and mounted onto the catheter or delivery device only just before the stent or stent-like device is inserted into the body lumen via the catheter or delivery device. In conventional methods for providing a stent or stent-like device for implantation, the stent or stent-like device is usually subjected to the necessary surface treatment in an expanded or semi-expanded state and subsequently compressed or crimped to a smaller diameter, which is suitable for mounting onto the catheter or delivery device and subsequent insertion into the body of a patient.

Many different devices and methods used for crimping a stent or stent-like device are well known from the prior art. When using a crimping device or method according to the prior art, the stents or stent-like devices are generally subjected to the surrounding environment in an unprotected fashion during the insertion into the crimping apparatus and are contacted by the elements of the crimping device. In the process, they are subjected to contamination by, e.g., reagents situated in the air, such as hydrocarbon molecules, which can adversely affect a hydrophilic surface of a stent, or an active substance on the stent, or stent-like device. The stent surface can also be contaminated by residues on the elements of the crimping device. Furthermore, there is the risk of an undesired contamination of or a change of the characteristics of the stent surface when transferring the stent or stent-like device to or a crimped stent or stent-like device from a conventional crimping device. Some methods even include manipulating the stent or stent-like device by hand to arrange the stent or stent-like device on a crimping device or at a catheter delivery system.

To simplify the procedure of preparing a stent for insertion into a body lumen, U.S. Pat. No. 8,834,550 B2 shows an assembly for loading and delivering a stent to a body lumen. The assembly includes a stent delivery catheter having a longitudinal inner member with a proximal handle and a tubular outer member coaxially arranged on the inner member and having a distal handle. The inner member and the outer member are slideable relative to each other. At its distal end, the outer member ends into a sleeve-like transfer member used for accommodating and compressing the stent. The distal end of the inner member extends from the outer member and through the transfer member. The stent is fixed to sutures which run from the stent through the delivery catheter to the proximal handle. When pulling the proximal handle relative to the distal handle, the sutures and the inner member slide axially relative to the outer member and the transfer member. The transfer member includes a funnel passage such that the stent is compressed when it slides along the passage and can be received within the outer member in a compressed state. The transfer member is removed and the stent is readily loaded into the delivery catheter for insertion into a body lumen. The sutures may also be removed. Although the assembly of U.S. Pat. No. 8,834,550 B2 facilitates the process of loading the stent into the delivery catheter the stent is constantly exposed to the environment and subject of re-contamination. A similar method for loading and delivering a stent is disclosed in EP 2018138 B1.

Further EP 1117349 B1 discloses an apparatus for loading a bag-enclosed stent into a catheter. The stent is packed in a flexible bag and the bag including the stent is arranged within a block including a conical inner passage. A catheter is connected to the block at an end of the passage. A pull string is attached to the bag and thread through the catheter. When pulling the pull string the bag including the stent is pulled into the catheter and the stent is compressed along the conical passage. The bag is equipped with a perforation, such that the bag can be removed from the stent by the pull string. Although the stent is fully protected by the closed bag, the process of providing the stent within the closed bag and arranging a pull string through the catheter and removing the bag from the stent within the catheter is cumbersome and complicates the preparation of the stent before application into a body lumen.

### SUMMARY

It is an objective of the present invention to propose an arrangement and a method for providing a self-expanding stent-like device for implantation into a body lumen, which prevent an adverse effect on or a contamination of the self-expanding stent-like device, more particularly the stent surface, during the storage and the radial compression of the self-expanding stent-like device, which simplify the handling of the self-expanding stent-like device during the preparation for the implantation and increase the assurance against undesired interactions of the stent surface before the implantation.

These and other objects are fulfilled by an arrangement and a method for storage of a self-expanding stent-like device according to the independent claims. Advantageous features and preferred embodiments of the arrangement and of the method according to the invention are disclosed in the dependent claims.

It is emphasized that an arrangement and a method according to the present invention is advantageous for storing all kinds of self-expanding, mainly sleeve-like or tube-like implants, that are compressed for implantation and delivered to an implantation spot by an elongated, tube-like delivery system. Therefore, the present invention refers to all such self-expanding stent-like devices, in particular including stents and stent-grafts provided for implantation. But also the present invention provides an advantageous storage arrangement for self-expanding stent-like devices that are not necessarily made for permanent implantation; for example, stent-like filters like a vena cava filter or stent retrievers. In the following, for an easier way of description of the inventive features of the invention, it is simply referred to as a stent, which shall represent all self-expanding stent-like devices in the context of this invention. Further, the arrangement and the method according to the present invention is in particular advantageous for storing a self-expanding stent-like device, which needs to expand over its entire length at an application site. Preferably, the stent or stent-like device has a hydrophilic surface.

According to the present invention, an arrangement for storage of a self-expanding stent-like device includes a container for storing the stent and a stent delivery system arranged at the container. In this connection, the term “arranged at” can relate to a directly or indirectly connected position of the stent delivery system in relation to the container. The arrangement can include the stent-like device. The stent delivery system includes an outer tube and an inner shaft axially moveable relative to the outer tube. The stent delivery system can, for example, be realized by an outer tube of a catheter and a guide wire as the inner shaft as generally used in the state of the art. The inner shaft is sufficiently stiff to be pushed and pulled back and forth within the outer tube.

The container includes an interior space advantageously being big enough for accommodating the stent in an expanded state or in an at least mostly expanded state. The container can be produced in one piece. Alternatively, the container can be assembled of two or more parts together establishing the interior space. Further, the container includes one stent passage opening as an access to the interior space for introducing or retrieving the stent. It is advantageous that the container includes only one stent passage opening because with this design it is easier to keep the interior space separated from the outside environment surrounding the container, as compared to a design with more than one stent passage opening. In addition, the stent passage opening can be used for filling the interior space of the container with a storage medium.

Preferably, the interior space is filled with an inert medium, which at least is inert in respect of a stent surface. Thus, the quality of the stent surface can be maintained, while the stent is stored in the container. This is particularly important for hydrophilic stents, of which the surfaces are highly purified and quickly become re-contaminated. Preferably, the container is made of a rigid material to provide protection for the stent against impact from outside the container. Further, a transparent material allows inspection of the stent while it is stored in the container.

Although the container includes only one stent passage opening for introducing or retrieving the stent, the container may have a further opening, e.g., for rinsing the container, filling the container with medium prior or after inserting the stent, controlling the pressure in the container after inserting the stent. Such further opening can, for example, include a thread and corresponding screw cap or a luer lock system to keep the container closed if no access to the interior space is needed.

A distal end of the outer tube of the stent delivery system is connected to the stent passage opening of the container. The term “distal” as used herein can relate to a direction towards a patient in an intended use of the respective component. For example, the distal end of the stent delivery system is its end intended to be directed to the patient when being applied.

By the outer tube or its distal end and the container or its stent passage opening being connected, the interior space of the container merges into the inner space of the outer tube. The distal end may be connected directly to the stent passage opening or it may be connected via a connection element to the stent passage opening. For example, it can be connected via a sleeve element as described in more detail below.

Located at or near the stent passage opening is an insertion part of ring-like shape. The term “near” in this connection can relate to a position comparably close to the stent passage opening. In embodiments in which the outer tube protrudes through the stent passage opening, the distal end of the outer tube can still be understood as being near the stent passage opening. The insertion part serves for compressing and inserting the stent into the outer tube of the delivery system as will be explained below.

The distal end of the outer tube can realize or be the insertion part, for example, the outermost edge of the outer tube, or it can be realized by a circumference of the stent passage opening of the container, like the circular edge of the opening. Alternatively, the insertion part can be realized by a separate sleeve element positioned at the stent passage opening and arranged at the distal end of the outer tube for connecting the outer tube to the stent passage opening of the container. Thus, the connection element as mentioned above can realize the insertion part as mentioned before.

In one preferred embodiment, the separate sleeve element is movable relative to the container. Thereby, the sleeve element can protrude into the interior of the container via the stent passage opening to a variable extent. This allows for adjusting the arrangement according to the situation given in a specific application. In another preferred embodiment, the separate sleeve element is formed as a portion of the container. The term “a portion of” as used herein can relate to two elements being integral with each other or to two elements being fixed to each other, e.g., screwed, bonded, glued, or clamped to each other or the like. Such a sleeve element can allow for providing robustness and convenient handling.

In a preferred embodiment, the container includes a body part and a cover part mounted to the body part. Thereby, the body part of the container preferably includes an open side which is closed by the cover part. Such multi-part embodiments of the container can allow for efficiently manufacturing the container. For example, particularly when the container is to be manufactured in an injection molding process, completely closed forms typically are difficult to realize. Therefore, having a container including plural parts allows for efficiently manufacturing the container by injection molding. Thereby, the separate sleeve element preferably is formed as a portion of the cover part or the body part of the container. This allows for efficiently manufacturing a robust cover part together with the sleeve element in one single manufacturing step.

Preferably, the body part of the container includes a first mount structure and the cover part of the container includes a second mount structure, which is complementary to the first mount structure of the body part. Thereby, the first mount structure of the body part and the second mount structure of the cover part can be arranged to fixedly mount the cover part to the body part. For example, such fixed mounting can be provided by equipping the first and second mount structures with teeth or ratchet teeth inter-engaging when being connected. Like this, a safe container can be efficiently provided. Alternatively, the first mount structure of the body part and the second mount structure of the cover part are arranged to releasably mount the cover part to the body part. For example, such releasable mount structures can be embodied as screw threads, wherein the container may form a male thread and the cap a female thread. Such releasable cover part allows for opening and accessing the interior of the container, which may be desired in some applications.

According to the invention, the inner shaft extends through the insertion part and includes a retainer for preventing the stent at least from axial movement relative to the inner shaft. In this connection, the term “preventing axial movement” relates to a connection between the stent and the inner shaft via the retainer in which essentially no movement of the inner shaft in relation to the stent along an axis of the inner shaft is possible. Essentially no movement does not exclude the stent having some clearance to a comparably small extent with regard to the inner shaft. However, the retainer more or less axially fixes the stent to the inner shaft. By providing such fixation, the stent can efficiently and conveniently be axially moved by operating the inner shaft. In particular, the stent can be moved by the inner shaft into and out of the insertion part, thereby compressing and expanding the stent.

The retainer can be embodied to radially extend into a web pattern or mesh of the stent, such that the stent is caught on the retainer by abutting at the retainer in longitudinal direction. In a possible embodiment, an outer profile of the retainer may include radial recesses for receiving the web pattern of the stent. For example, the retainer may be designed as a socket attached to the inner shaft and equipped with hooks or similar elements hinged into the web pattern or mesh of the stent. Thereby, an axial clearance may still exist by the hooks moving within the single mesh sections. In another embodiment, the outer profile of the retainer may include circumferential cuttings for receiving a web pattern of the stent for also preventing the stent from movement in radial direction relative to the inner shaft. For example, the retainer can be a deformable sleeve clamped on the inner shaft, wherein the stent is pressed into the sleeve such that it is press fitted to the sleeve. Of course, many other forms of retainers are possible which allow for achieving the intended effect of connecting the stent to the inner shaft.

In one embodiment, the inner shaft may extend into the container. In another embodiment, the inner shaft terminates at the stent passage opening or at least shortly behind it, and the retainer may be arranged at an end region of the inner shaft or at the tip of the inner shaft. When the stent is stored in the interior space of the container, a proximal end of the stent can be radially compressed onto the retainer and engages with the retainer such that at least an axial movement of the stent relative to the retainer is blocked.

In the first example, the retainer does not affect a radial compression or expansion movement of the stent structure during compression or expansion of the stent. The proximal end of the stent can be held in axial engagement with the retainer by the insertion part, which in this case encircles the proximal end of the stent opposite the retainer.

In the first example, when the stent is stored in the container the proximal end of the stent is compressed to a diameter smaller than a diameter of the ring-shaped insertion part and is held radially compressed by the insertion part in that the insertion part surrounds the compressed proximal end of the stent. That means that the insertion part axially extends over the retainer and overlaps with the retainer. At the same time, the retainer engages into the stent to prevent axial movement of the stent along the inner shaft. To do so, a radial extension of the retainer corresponds to the inner diameter of the insertion part, such that engagement into the stent is possible when the stent is held in compressed state by the insertion part. Thus, the retainer and the insertion part safely grasp the proximal end of the stent.

In the second example, the retainer additionally guarantees that the proximal end of the stent is kept radially compressed on the retainer in that the web pattern of the stent radially engages with the retainer. In this case, the insertion part can be axially distanced from the retainer and is not necessarily required to keep the proximal end of the stent in the compressed state.

In case of a self-expanding stent-like device not used for permanent implantation, i.e., that needs to be retracted from the application site after a treatment of the site, the proximal end of the stent-like device does not necessarily need to be released from the inner shaft. This is the case for example for stent retriever devices, the proximal end of which is not required to expand on the application site. Therefore, the proximal end can, for example, be permanently attached to the inner shaft or the retainer.

In case of a self-expanding stent-like device that is not used for permanent or long-term implantation, i.e., which needs to have the ability to be easily retracted from the application site after a certain amount of time, e.g., from a blood vessel, the proximal end of the stent-like device can permanently be attached to the retainer while the retainer can be released from the inner shaft. In case of the need to retrieve the stent-like device from the application site, the retainer can be captured by the inner shaft, for example via a hook-like mechanism, and retracted into the outer shaft of the stent delivery device. This is the case, for example, for stent-like filters, such as vena cava filters, which shall be able to be retrieved, and the expansion of the proximal end is not crucial for the function of the stent-like device.

For all examples, it is advantageous that in the compressed state of the proximal end the stent or stent-like device is readily prepared to be loaded into the outer tube, while the majority of a length of the stent remains in an at least mostly, preferably fully relaxed expanded state and only the proximal end of the stent is compressed already.

The insertion part may fulfill two functions. Its main function is to compress the stent when the stent is moved in direction of the outer tube. The stent is inserted into the outer tube or a sleeve element connected to the outer tube in that it is squeezed through the ring-shape of the insertion part. Further, the insertion part may function as a securing element that secures the engagement of the stent on the retainer as described for the first example of the retainer.

The arrangement according to the invention furthermore ensures that a surface area of elements of the delivery system which surface area is present within the container is reduced to a minimum and pollution of the interior space by the delivery system is nearly excluded.

In one embodiment, the inner diameter of the ring-like insertion part corresponds to the outer diameter of a stent compressed for application into a body lumen. For example, this is the case when the insertion part is realized by the outer tube. In another embodiment, the inner diameter of the ring-like insertion part is smaller than a diameter of the stent in an expanded state but larger than a diameter of a stent compressed for application into a body lumen. In this case, the retainer needs to be large enough to engage radially into the stent but also must be compressible together with the stent, when the stent is compressed for being loaded into the outer tube. For example, such compression can be accomplished by pulling the stent along a passage with a funnel surface that ends up in the outer tube. Such compression process is known from the prior art. In any case, a stent or stent area in a compressed state arranged inside the insertion part will be secured in its compressed state on the retainer.

The stent can easily be loaded into the outer tube and released from the outer tube again by pulling or pushing the inner shaft in one or the opposite direction. This can be used to introduce the stent into the container and retrieve it from the container. For releasing the stent at an application site in a body lumen, the outer tube of the delivery system is pulled back towards proximal direction. During the release process at the application site of the body lumen, the stent can even be loaded again into the outer tube of the delivery system, for example, if the stent is not located at the right spot, by pushing the outer tube of the delivery system into distal direction over the stent again. This can be performed as long as the proximal end of the stent is in a compressed state arranged inside the outer tube of the delivery system and secured in its compressed state on the retainer.

In case of a retainer according to the first example as mentioned above, as soon as the outer tube is fully pulled back in proximal direction such that the retainer is outside the distal end of the outer tube, the stent jumps off from the retainer due to its radial outward force which is based on the stent's elasticity and memory effect. Alternatively, the inner shaft may be pushed forward in distal direction relative to the outer tube to release the stent from the retainer. In case of a retainer according to the second example as mentioned above, wherein the retainer hinders radial expansion of the proximal end of the stent, the stent may be released from the retainer by a rotational movement or by an electrically induced release as known from the prior art.

The arrangement of the current invention has the advantage that only a small portion of the delivery system is located in the interior space of the container and exposed to the inert filling. Therefore, the risk of potential solvents eluting from the delivery system into the inert filling and onto the stent is reduced to a minimum. In addition, the majority of the stent is expanded and exposed to the inert filling. In case a sleeve element is used as insertion part or the insertion part is realized by the stent passage opening, the stent is not in direct physical contact at all with its delivery system but rather with packaging material or auxiliary elements that can easily be made of material compatible to the inert filling for the stent.

The arrangement for storage of a self-expanding stent according to the invention is advantageous for storing a stent in a safe environment. But even more the arrangement can be used for a method for storing the stent and subsequently preparing the stent for delivery into a body lumen according the present invention. In the beginning, a surface of the stent may be purified, in particular by a treatment for increasing the hydrophilicity of the stent surface. Also the stent delivery system is cleaned. According to the method of the invention, in a contamination free or low-contamination environment, at least the proximal end of the stent is compressed and the proximal end of the stent is compressed onto the retainer provided on the inner shaft. The proximal end of the stent is held in engagement with the retainer by the ring-like insertion part placed over the compressed proximal end of the stent and over the retainer or by the retainer itself as explained above. This compression step allows for two alternatives: i) only the proximal end of the stent is compressed and placed on the retainer, while the majority of the length of the stent remains in an expanded state; or ii) the whole stent is compressed, while the proximal end of the stent is placed on the retainer. For a full compression of the stent, advantageously the stent may be pulled into the outer tube by the pulling the inner shaft as explained before. In one or the other of the compressed states of the stent, the retainer engages into the proximal end of the stent and the stent is held in engagement by the insertion part. This state can be called the engaged state of the stent, in which the stent now is introduced into the container through the stent passage opening. The size of the stent passage opening determines if the stent is inserted in a fully compressed state or in a state where only the proximal end of the stent is compressed and the rest of the stent is expanded. The container is filled with a storage medium, preferably an inert filling as mentioned above. Now the stent is safely stored in the container and protected against recontamination. The arrangement of stent, container and delivery system in this condition, a storage state, serves as a packaging for the stent, wherein the stent can be stored for a longer period of time.

For preparing the stent for delivery into a body lumen, i.e., loading the stent into the delivery system, the retainer is retracted along the outer tube by pulling the inner shaft relative to the outer tube for compressing the stent and inserting the stent into the outer tube, while the stent is in the storage medium all the time. The compression of the stent is accomplished in that the expanded part of the stent is squeezed through a section of the insertion part with a narrow diameter. The narrow diameter corresponds to the diameter of the outer tube or can be slightly smaller so that the stent can be inserted into the outer tube.

An insertion part section with a funnel passage may be used to squeeze the stent. But it is emphasized that it is an advantage of the arrangement of the present invention that such a funnel passage is not always required. In case the self-expanding stent either is a bare metal stent with a web pattern including a closed cell design, e g, manufactured from a slotted, mostly laser cut tube, or is a stent with a web pattern of a braided design, the ring-edge of the ring-shaped insertion part with a narrow diameter is sufficient to compress the stent. The diameter of the ring-edge may correspond to the diameter of the outer tube and therefore the outer tube can be used as the insertion part. This reduces the number of required elements for the arrangement and reduces the number of elements the stent has to get in contact with while being prepared for delivery into the body lumen.

In one embodiment the arrangement according to the invention includes a first sealing between the stent passage opening of the container and the insertion part. The first sealing is used to fully seal off the interior space of the container relative to the environment outside of the container. The first sealing can be a ring-seal positioned around the insertion part, e.g., represented by the outer tube or a sleeve element, and brought into contact with the container. For example, the ring-seal can be pushed along the outer tube or the sleeve element until it abuts against the container. For arranging the outer tube or a sleeve element at the container, e.g., when inserting the stent into the container as mentioned above, the first sealing can be in place at the container already. The outer tube or a sleeve element may be pushed through the first sealing and at the same time tight sealing is established. Alternatively, first the outer tube or a sleeve element may be positioned within the stent passage opening and the first sealing is placed in a tight sealing position afterwards. A specific container design for receiving the ring-seal in tight sealing contact is presented in the figures described below. Alternatively the first sealing can be realized by a tight connection between container and outer tube. For example, the material of the outer tube is slightly elastic so that the outer tube can seal off with the surface of the container.

Further, the outer tube and the inner shaft can be sealed to each other by a second sealing. Thereby, the second sealing can be provided inside the outer tube between the outer tube and the inner shaft. Again, a ring-seal can be used. As explained above, the interior space of the container merges with the interior of the outer tube. Thus, the outer tube may allow access to the interior space of the container. The second sealing prevents the storage medium from escaping through the outer tube.

Of course sealings can also have cylindrical shape. The second sealing can also be a cover which covers the outer tube on its proximal end and which has an opening, through which the inner shaft may protrude. This opening is tight enough to enable desired sealing and to prevent leakage from the container. A possible material for the sealings is rubber or any other kind of elastic polymer.

In one embodiment of the arrangement according to the invention, a stopper is arranged inside the outer tube at a distance to the distal end of the outer tube, which is at least slightly larger than a length of the stent in a fully compressed state. Thus, the stent in the fully compressed state can be covered by the outer tube completely. The stopper for example is realized by protrusions on the inner surface of the outer tube extending radially into the outer tube. Thus the stent, when being inserted by pulling the inner shaft, will be blocked at the stoppers as soon as it is fully compressed. The blocking of the insertion movement is an indication to the user that the stent is fully loaded into the delivery system.

Further, it is an advantage of the method and the arrangement according to the current invention that the stent can be easily introduced into the container without the risk of recontamination of the stent surface after it had been purified. This is achieved in that, after compressing the proximal end of the stent onto the retainer and placing the insertion part over the compressed proximal end, the retainer is retracted into the outer tube by pulling the inner shaft relative to the outer tube for loading the stent into the outer tube. In this stent loaded state, the outer tube is arranged at the stent passage opening and the stent is inserted into the container by pushing the retainer forward within the outer tube until only the proximal end of the stent remains compressed by the insertion part placed over the compressed proximal end. By releasing the majority of the length of the stent from the outer tube into the container, the major part of the stent expands into an expanded state within the container. In case a ring-seal is used as the first sealing, it can be placed in contact with the container before or after releasing the stent into the container.

On the other hand, when using the arrangement for storage of the stent according to the present invention, the delivery system is ready for application of the stent in just one action. The medical practitioner solely needs to pull the inner shaft and only in one direction. By pulling the inner shaft, first the stent is compressed and inserted into the outer tube of the delivery system and second, when the stent is fully loaded and further movement is blocked by the stopper, continuous pulling of the inner shaft results in detaching the outer tube from the container or the sleeve element, respectively. The delivery system including the loaded stent is ready to deliver the stent to an application site.

### BRIEF DESCRIPTION OF THE DRAWINGS

Several example embodiments of the invention will be illustrated in the following drawings, which merely serve for explanation and should not be construed as being restrictive. The features of the invention becoming obvious from the drawings should be considered to be part of the disclosure of the invention, both on their own and in any combination. In the drawings:

FIG. 1a shows a first version of a first embodiment of an arrangement for storing a self-expanding stent according to the present invention including an insertion part realized by a distal end of an outer tube of a delivery system;

FIG. 1b shows a second version of a first embodiment of an arrangement for storing a self-expanding stent according to the present invention including an insertion part realized by a distal end of an outer tube of a delivery system and a container with an additional opening;

FIG. 1c shows a third version of a first embodiment of an arrangement for storing a self-expanding stent according to the present invention including an insertion part realized by a distal end of an outer tube of a delivery system and a container with a body part and a cover part having a stent passage opening;

FIG. 1d shows a fourth version of a first embodiment of an arrangement for storing a self-expanding stent according to the present invention including an insertion part realized by a distal end of an outer tube of a delivery system and a container with a body part having an additional opening and a cover part having a stent passage opening;

FIG. 1e shows a fifth version of a first embodiment of an arrangement for storing a self-expanding stent according to the present invention including an insertion part realized by a distal end of an outer tube of a delivery system and a container with a cover part and a body part having a stent passage opening;

FIG. 2a shows a first version of a second embodiment of an arrangement for storing a self-expanding stent according to the present invention including an insertion part realized by a separate sleeve element movable relative to a container;

FIG. 2b shows a second version of a second embodiment of an arrangement for storing a self-expanding stent according to the present invention including an insertion part realized by a separate sleeve element movable relative to a cover part of a container;

FIG. 2c shows a cover part of a container of a third version of a second embodiment of an arrangement for storing a self-expanding stent according to the present invention including an insertion part realized by a separate sleeve element integral with the cover part of the container;

FIG. 2d shows a cover part of a container of a fourth version of a second embodiment of an arrangement for storing a self-expanding stent according to the present invention including an insertion part realized by a separate sleeve element integral with the cover part of the container and extending into an interior of the container;

FIG. 2e shows a fifth version of a second embodiment of an arrangement for storing a self-expanding stent according to the present invention including an insertion part realized by a separate sleeve element movable relative to a body part of a container;

FIG. 3a shows a first version of a third embodiment of an arrangement for storing a self-expanding stent according to the present invention including an insertion part realized by a circumferential section of a stent passage opening embodied at the end of a separate sleeve element integral with the container;

FIG. 3b shows a second version of a third embodiment of an arrangement for storing a self-expanding stent according to the present invention including a container with a body part, a cover part and an insertion part realized by a circumferential section of a stent passage opening embodied at the end of a separate sleeve element integral with the cover part;

FIG. 3c shows the cover part of the second version of FIG. 3b;

FIG. 3d shows a cover part of a container of a third version of a third embodiment of an arrangement for storing a self-expanding stent according to the present invention including an insertion part realized by a separate sleeve element integral with the cover part of the container and extending into an interior of the container;

FIG. 3e shows a fourth version of a third embodiment of an arrangement for storing a self-expanding stent according to the present invention including a container with a body part, a cover part and an insertion part realized by a circumferential section of a stent passage opening embodied at the end of a separate sleeve element integral with the body part;

FIG. 3f shows a fifth version of a third embodiment of an arrangement for storing a self-expanding stent according to the present invention including a container with a body part, a cover part and an insertion part realized by a circumferential section of a stent passage opening embodied at the end of a separate sleeve element integral with the body part of the container and extending into an interior of the container;

FIG. 4a shows a first version of a fourth embodiment of an arrangement for storing a self-expanding stent according to the present invention including an insertion part realized by a circumferential section of a stent passage opening embodied at the end of a separate sleeve element integral with the container and a first sealing;

FIG. 4b shows a second version of a fourth embodiment of an arrangement for storing a self-expanding stent according to the present invention including an insertion part realized by a circumferential section of a stent passage opening embodied at the end of a separate sleeve element integral with the body part, a first sealing, and a container with a cover part and a body part having the stent passage opening;

FIG. 4c shows a third version of a second version of a fourth embodiment of an arrangement for storing a self-expanding stent according to the present invention including an insertion part realized by a circumferential section of a stent passage opening embodied at the end of a separate sleeve element integral with the body part, a first sealing, and a container with a cover part and a body part having the stent passage opening, wherein the circumferential section of the stent passage opening extends into an interior of the container;

FIG. 5a shows a first version of a fifth embodiment of an arrangement for storing a self-expanding stent according to the present invention including an insertion part realized by a separate sleeve element movable relative to a container and an enlarged stent passage opening with a first sealing;

FIG. 5b shows a second version of a fifth embodiment of an arrangement for storing a self-expanding stent according to the present invention including an insertion part realized by a separate sleeve element movable relative to a container and an enlarged stent passage opening with a first sealing including a modified retainer;

FIG. 6 shows a sixth embodiment of an arrangement for storing a self-expanding stent according to the present invention including an insertion part realized by a distal end of an outer tube of a delivery system and an enlarged stent passage opening with a first sealing;

FIG. 7a shows a schematic illustration of a stent loading step when using the first embodiment of an arrangement for storing a self-expanding stent according to FIG. 1;

FIG. 7b shows a schematic illustration of a detaching step detaching the delivery system from the container after the stent loading step of FIG. 7a;

FIG. 7c shows a schematic illustration of using the delivery system after the detaching step of FIG. 7b;

FIG. 8a shows a schematic illustration of a web pattern of a self-expanding stent including closed cells;

FIG. 8b shows a schematic illustration of a web pattern of a self-expanding stent including open cells;

FIG. 8c shows a schematic illustration of a web pattern of a self-expanding stent including a braided design with closed cells;

FIG. 9a shows a schematic illustration of a first example of a stopper inside an outer tube of a delivery system;

FIG. 9b shows a schematic illustration of a second example of a stopper inside an outer tube of a delivery system;

FIG. 9c shows a schematic illustration of a third example of a stopper inside an outer tube of a delivery system;

FIG. 10a shows a schematic illustration of a step of an alternative stent preparing method when using an arrangement for storing a self-expanding stent including a separate sleeve element movable relative to a container;

FIG. 10b shows a schematic illustration of a subsequent step of the alternative stent preparing method after the step of FIG. 10a;

FIG. 11a shows a first version of a proximal end of an outer tube of a stent delivery system as it is embodied in all embodiments of arrangements for storing a self-expanding stent according to the present invention shown in FIGS. 1 to 6 equipped with a second sealing;

FIG. 11b shows an alternative second version of a proximal end of an outer tube of a stent delivery system equipped with a second sealing and a shrink tube;

FIG. 11c shows an alternative third version of a proximal end of an outer tube of a stent delivery system equipped with a second sealing, a shrink tube and an additional second sealing; and

FIG. 11d shows an alternative second version of a proximal end of an outer tube of a stent delivery system equipped with a second sealing and an additional second sealing included in a Tuohy Borst sealing adapter.

### DETAILED DESCRIPTION

In the following description, certain terms are used for reasons of convenience and are not intended to limit the invention. Terms like “right”, “left”, “up”, “down”, “under” and “above” refer to directions in the figures. The terminology includes the explicitly mentioned terms as well as their derivations and terms with a similar meaning. In addition, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the example term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

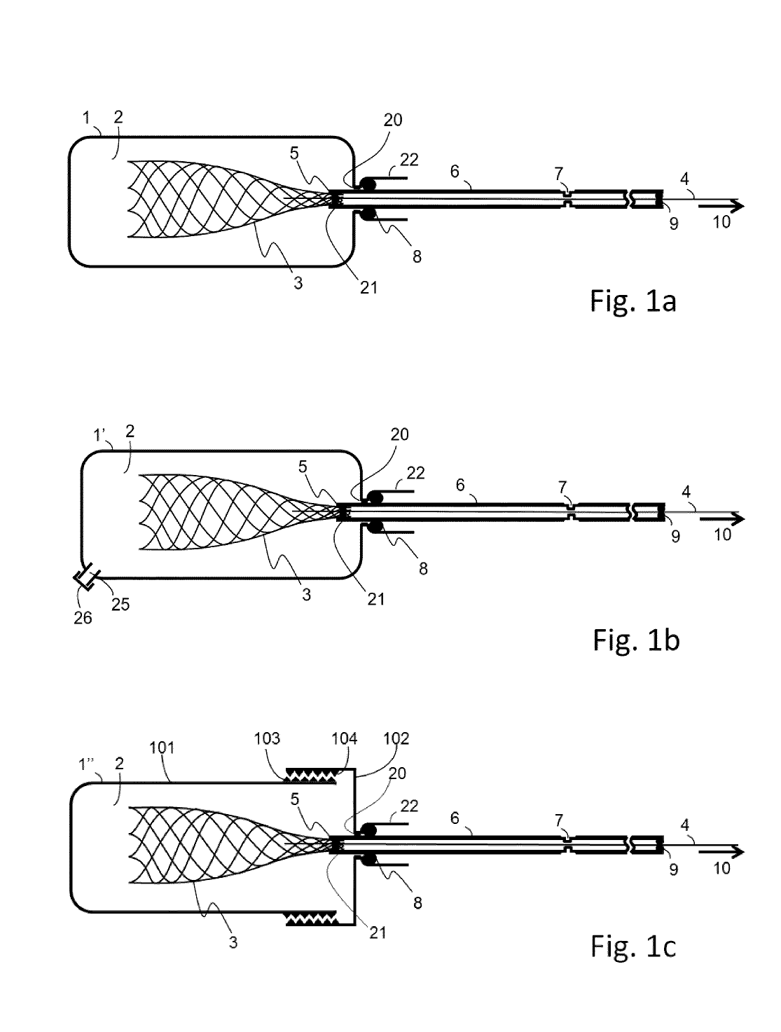

More particular, FIGS. 1 to 6 show different embodiments of an arrangement for storage of a self-expanding stent according to the present invention including different examples of an insertion part as will be explained below. The like elements of the arrangements in the different embodiments will be marked with the same reference numbers. It should be understood that technical characteristics described for one embodiment also can apply to another embodiment and therefore are not necessarily repeated in the description of each embodiment.

FIGS. 1 to 6 illustrate elements of an arrangement for storage of a self-expanding stent 3 in a storage state storing the stent 3 in a container 1 by way of a schematic longitudinal cut through the arrangement. The container 1 includes an interior space 2, which is large enough to store the stent 3 in an expanded condition. The container 1 includes one stent passage opening 20 as an access to the interior space 2 of the container 1. The container 1 is made of a rather rigid material, which is sufficiently bend-proof, such that the container cannot collapse and damage the stent 3. Further, the material of the container 1 should not react in contact with a storage medium with which the interior space might be filled for conservation of the stent, as will be explained below. The container 1 can, for example, be made of glass, which not only provides proper protection from influences from outside the container and does not interact with most of the available storage medium. In addition, glass is transparent and therefore offers the option of visual examination of the stent condition inside the container. Other suitable materials are, e.g., polymer materials, in particular transparent polymer materials. The container 1 as shown in most of the figures is designed either as a one-piece container or comprised of two or more separately produced container parts that are tightly connected with each other, for example by gluing, or molding, or by a mechanical connection.

The container 1 in FIG. 1a is shown as a container with only one opening, namely the stent passage opening 20. In contrast, the container 1′ in FIG. 1b includes a further opening 25 sealed by a cap 26. The further opening 25 can be used as an access to the interior space 2 without having the need to move the stent. For example, the access can be used for flushing the interior space with a specific medium, for controlling the pressure in the interior space, etcetera.

As another alternative, the container 1″ of FIG. 1c includes a body part 101 and a cover part 102. The body part 101 has a completely open side; in FIG. 1c this is the right-hand side, which is closed by the cover part 102. Near to the open end, the body part 101 is equipped with outer circumferential teeth 103 as first mount structure. The cover part 102 has corresponding inner circumferential teeth 104 as second mount structure, which engage the outer circumferential teeth 103 of the body part 101. Like this, the cover part 102 is tightly fixed to the body part 101. The cover part 102 further includes the stent passage opening 20 for accessing the interior space 2 of the container 1″.

As a further other alternative, the container 1′″ of FIG. 1d includes a body part 101′ and a cover part 102 similar as described hereinbefore. However, in contrast to the body part 101 described above, the body part 101′ is further equipped with a further opening 25, which can be used as an access to the interior space 2, sealed by a cap 26.

As still another alternative, the container 1″″ of FIG. 1e includes a body part 101″ and a cover part 102′. However, in contrast to the containers 1″ and 1′″ described above, the cover part 102′ is completely closed and the stent passage opening 20 for accessing the interior space 2 of the container 1″″ is comprised by the body part 101″.

Further, the arrangement comprises a delivery system, which is designed to deliver a stent to an application site as explained in the introductory section of the present description of the invention. The delivery system includes an inner shaft 4 and an outer tube 6. For example, a guide wire as it is used for neurovascular, intracranial stents, and/or flow diverters can be used as the inner shaft 4. In other delivery systems for self-expanding stents, the inner shaft is realized as a tube element and a guide wire will run through this tube element during intervention and delivery of the stent into a body lumen. In this case, the guide wire has been previously placed into the body lumen.

The inner shaft 4 is slideably arranged within the outer tube 6. At its distal end, the inner shaft includes a retainer 5 for preventing the stent 3 from axial movement relative to the inner shaft 4, as will be explained in more detail below. At its proximal end, the inner shaft 4 includes an actuation member (not shown), which is used for pulling and pushing the inner shaft 4 relative to the outer tube 6 in a longitudinal direction. It will be understood by a person skilled in the art that the delivery system may have more elements running in longitudinal direction of the system as is known from the prior art. All such delivery systems can be used for the arrangement of the present invention as long as they include an outer tube 6 and an inner shaft 4 as described herein.

The embodiments of the arrangement as illustrated in FIGS. 1a to 4c particularly distinguish in their design of an insertion part of ring-like shape, which is located at or near the stent passage opening 20. It is common to all different insertion parts that, when the stent is stored in the interior space of the container as shown, a proximal end of the stent is compressed to a diameter smaller than a diameter of the insertion part and is held radially compressed by the insertion part. The radial compression urges the proximal end of the stent 3 into axial engagement with the retainer 5, which engagement inhibits an axial movement of the stent 3 relative to the inner shaft 4. Thus, the stent 3 can move back and forth in the longitudinal direction relative to the outer tube 6, the insertion part and the container 1 by actuation of the inner shaft 4 within the outer tube 6.

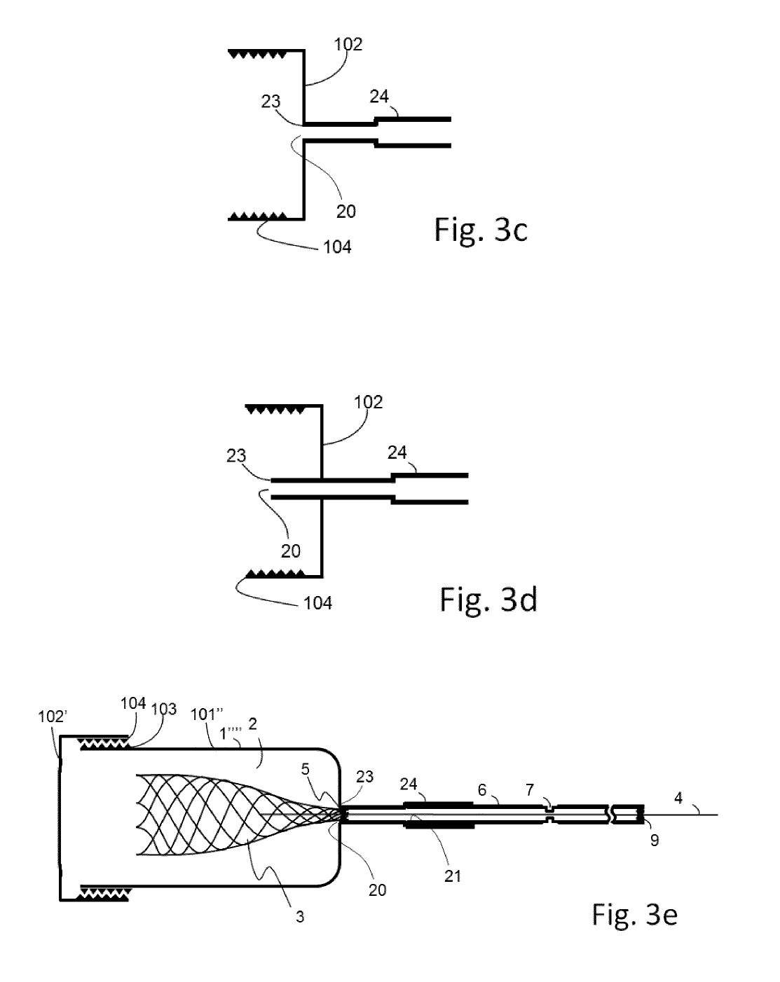

To ensure a reliable form fit between stent 3, retainer 5, and insertion part, the ring-shaped insertion part encloses the retainer 5 and presses a web pattern of the stent 3 into engagement with the retainer 5. The inner diameter of the insertion part and the outer diameter of the retainer 5 are coordinated for this function. However, for the arrangement as illustrated in FIGS. 1a to 4c, 5a, and 6, the web pattern of the stent 3 does not require to engage with the retainer 5 in radial direction to ensure that the stent can easily relax and expand in radial direction. An alternative example of holding the proximal end of the stent in radial compression on the retainer will be discussed for the embodiment shown in FIG. 5b.

In the versions of the first embodiment shown in FIGS. 1a to 1e, the insertion part is realized by a distal end 21 of the outer tube 6. The distal end 21 is pushed through the stent passage opening 20 of the container 1 and terminates near the stent passage opening 20, that is shortly behind the stent passage opening 20. The distal end 21 is positioned radially over the retainer 5 such that the compressed proximal end of the stent 3 is pressed into engagement with the retainer 5. Thus, the insertion part in form of the distal end 21 of the outer tube 6 prevents the proximal end of the stent 3 from radial movement and the retainer prevents the proximal end of the stent 3 from axial movement. The remaining part of the stent, which is the majority of the length of the stent, remains in an expanded condition. In this condition, i.e., the storage state of the arrangement, the stent can be stored for a long period of time before it needs to be prepared for implantation into a body lumen. Thus, the arrangement according to the invention serves as a packaging for the self-expanding stent.

The container is preferably filled with a storage medium in form of an inert filling that has an inert property in respect of the stent surface and therefore maintains a high purification level, in particular it preserves a hydrophilic characteristic of the stent. The container can be filled with the inert filling before or after the stent is placed into the container. The arrangement includes a first sealing 8 between the stent passage opening 20 of the container 3 and the insertion part to seal off the interior space of the container against the environment around the arrangement. The first sealing 8 is provided by a ring-seal, which sits tight on the outer surface of the outer tube 6. As a counter part for a tight fit of the ring-seal on the outer tube, the container includes an integral reception sleeve 22 extending around the stent passage opening 20. The reception sleeve 22 is designed to encompass the ring-seal such that the ring-seal is slightly pressed in between the outer surface of the outer tube 6 and the inner surface of the reception sleeve 22. To put the ring-seal in place, it simply can be pushed manually along the outer tube 6 into the reception sleeve 22.

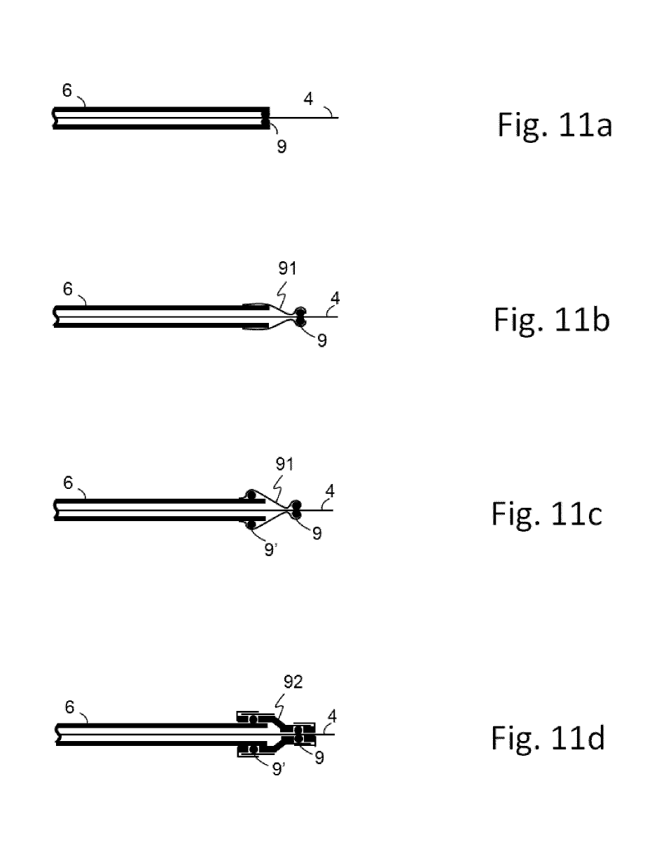

A second sealing 9 is provided inside the outer tube 6 between the outer tube 6 and the inner shaft 4. The second sealing 9 seals off the interior space 2 against the inner volume of the outer tube 6. Because the distal end 21 of the outer tube 6 is open, the interior space 2 and the interior volume of the outer tube 6 are joined. To limit the overall volume of interior space 2 and inner volume of outer tube 6, the second sealing 9 acts as a barrier and the inert filling cannot leak out of the interior space 2 through the inner volume of the outer tube 6. Furthermore, no contaminating particles can enter the interior space 2 through the outer tube 6. The second sealing 9 can be provided by a ring-seal of suitable size for a tight fit with the inner surface of the outer tube and the outer surface of the inner shaft 4, but allows the inner shaft 4 to slide through. Other designs of arrangements for sealing the outer tube 6 and the inner shaft 4 to each other are described in FIGS. 11a to 11d.

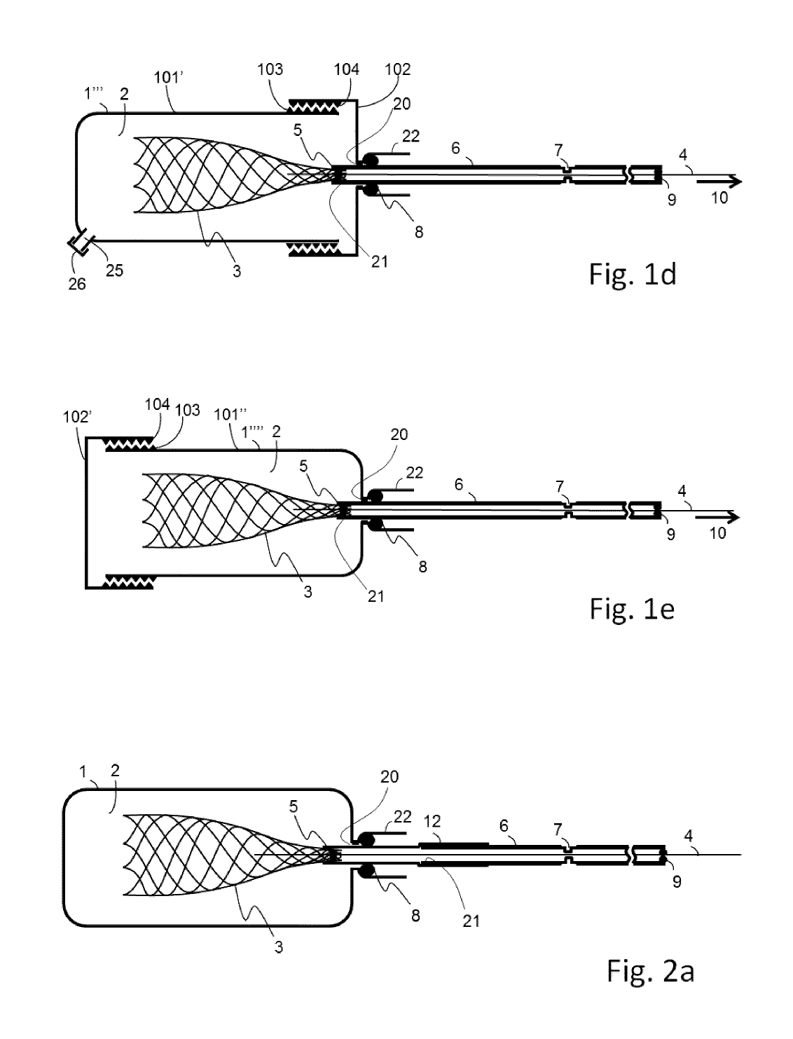

The embodiments of arrangements according to the invention shown in FIGS. 2, 3, 4, and 10 include a separate sleeve element. Thereby, embodied as sleeve element 12, 12′, the separate sleeve element is movable relative to the container. Or, embodied as insertion sleeve 24, the separate sleeve element is integral with the container.

In the several versions of the second embodiment exemplified in FIGS. 2a to 2e, the insertion part is realized by the separate sleeve element 12, 24. The separate sleeve element 12, 24 can take several forms and can cooperate with the container in respectively different ways. In a first version of the second embodiment of FIG. 2a, the insertion part is realized by a sleeve element 12 as a separate sleeve element. In this case, the separate sleeve element 12 is movable relative to the container 1. The sleeve element 12 also serves as a connecting element for connecting the outer tube 6 with the stent passage opening 20 of the container. The distal part of the sleeve element 12 is pushed through the stent passage opening 20 of container 1 and terminates near the stent passage opening 20, namely in this case shortly behind the stent passage opening 20. Further, the distal part of the sleeve element is positioned radially over the retainer 5 such that the compressed proximal end of the stent 3 is pressed into engagement with the retainer 5. The proximal part of the sleeve element 12 is designed to receive the distal end 21 of the outer tube 6. To do so, the proximal part of the sleeve element 12 includes a slightly larger diameter such that, when the outer tube 6 is positioned within the proximal part, the inner surface of the outer tube 6 is flush with the inner surface of the distal part of the sleeve element 12. Therefore, when the stent is pulled into the sleeve element 12 by pulling the inner shaft 4, the stent slides smoothly from the distal part of the sleeve element 12 into the outer tube 6. The material of the sleeve element 12 can be chosen to be compatible with the storage medium, in particular with the inert filling.

In a second version of the second embodiment exemplified in FIG. 2b, while the overall functioning remains analogous to that of FIG. 2a, the container 1″ includes a body part 101 and a cover part 102 mounted on the body part having a stent passage opening 20, as described in connection with FIG. 1c, for instance. As a counter part for a tight fit of the ring-seal 8 on the outer tube, the cover part 102 of the container 1″ includes an integral reception sleeve 22 extending around the stent passage opening 20, as already described in connection with FIGS. 1a-1e. The sleeve element 12 is in this case movable relative to the cover part 102 of the container 1″. The first sealing 8 is slightly pressed in between the outer surface of the sleeve element and the inner surface of the reception sleeve 22 such that a tight connection is provided.

In a third and in a fourth version of the second embodiment of the arrangement for storing a self-expanding stent according to the present invention, exemplified in FIGS. 2c and 2d, the insertion part is realized by a separate sleeve element in the form of an insertion sleeve 24, wherein the insertion sleeve 24 is integral with the cover part 102 of the container 1. In both cases, the cover part 102 integrally includes also a reception sleeve 22′, substantially adjacent to the insertion sleeve 24. The reception sleeve 22′ is also in this instance designed to encompass a first sealing 8, for example a ring-seal, such that the ring-seal is slightly pressed in between the outer surface of the outer tube 6 and the inner surface of the reception sleeve 22. In the specific case of FIG. 2d, the insertion sleeve 24 extends into an interior of the container, the extension forming a circular edge 23 of the stent passage opening 20 which projects beyond a surface of the cover part 102 internal to the container. In FIG. 2c, an extremity of the insertion sleeve opens into the cover part 102 to form a circular edge 23 of the stent passage opening 20.

In a fifth version of the second embodiment exemplified in FIG. 2e, the positioning of the body part 101″ and the cover part 102′ mounted thereon in the container 1″″ is inverted with respect to that of FIG. 2b. The sleeve element 12 is thus designed to be movable relative to the body part 101″ of the container 1″″. The stent passage opening 20 is in this case incorporated in the body part 101″. In addition, the body part 101″ of the container 1″″ is provided with a reception sleeve 22 extending around the stent passage opening 20, to keep a ring-seal 8 in place against the outer tube 6. The reception sleeve is integral with the body part 101″. As in the embodiment of FIG. 1, the embodiment of FIGS. 2a, 2b, and 2e are shown in the storage state of the arrangement. In particular, the embodiment of FIGS. 2a to 2e also includes a first sealing 8 and a second sealing 9.

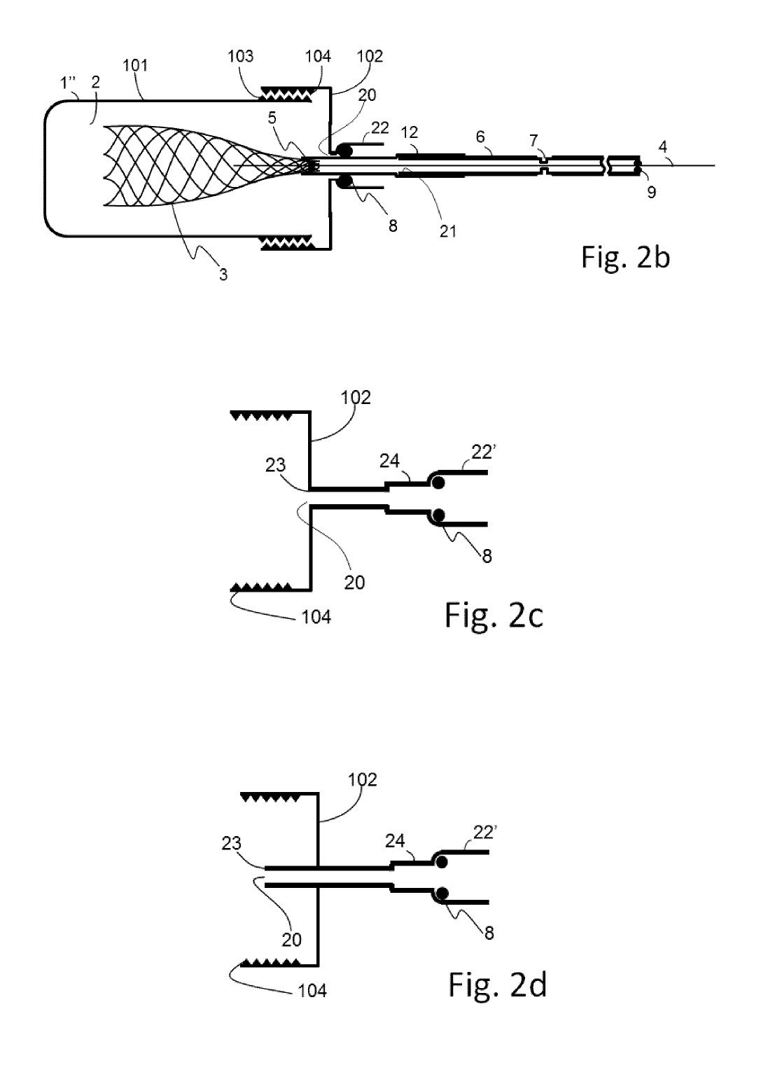

In several versions of a third embodiment of FIGS. 3a to 3f, the insertion part is realized by a circumference of the stent passage opening, for example by a circular edge 23 of the stent passage opening 20.

In a first version of the third embodiment, represented in FIG. 3a, the container 1 includes an insertion sleeve 24 as a separate sleeve element surrounding the stent passage opening 20 and extending axially from the circular edge 23, for directly connecting the outer tube 6 to the stent passage opening. The inner shaft 4 carrying the retainer 5 is axially positioned within the stent passage opening such that the circumference of the stent passage opening is located radially over the retainer 5 such that the compressed proximal end of the stent 3 is pressed into engagement with the retainer 5. The proximal part of the insertion sleeve 24 is designed to receive the distal end 21 of the outer tube 6, analogue to the proximal part of the sleeve element 12 shown in FIG. 2a. Thus, the inner surface of the outer tube 6 is flush with the inner surface of the distal part of the insertion sleeve 24. The size of the circumference of the stent passage opening and the circular edge 23, respectively, is coordinated with the outer dimension of the retainer 5 so that the stent 3 can be held in place on the retainer 5.

In a second version of the third embodiment, shown in FIG. 3b, a container 1″ includes a body part 101 and a cover part 102. The cover part 102 integrally includes an insertion sleeve 24 as a separate sleeve element. The insertion sleeve is separate from the outer tube 6, from the inner shaft 4 and from the stent-like device 3. The insertion part is realized by a circumferential section or edge 23 of the stent passage opening 20 which is embodied at the distal end of the insertion sleeve 24. In FIG. 3c, the cover part 102 of the arrangement of FIG. 3b is shown, wherein it is evident how the sleeve element 24 is integral with the cover part 102 and how cover part and sleeve element cooperate to create an insertion part at the stent passage opening 23.

FIG. 3d shows the cover part 102 of a container compatible with a third version of the third embodiment of the arrangement according to the present invention. In short, the cover part 102 is modified with respect to that of FIG. 3c in that the insertion sleeve 24 as a separate sleeve element extends into an interior of the container by protruding beyond an internal surface of the cover part 102. The circumferential section 23 of the stent passage opening 20, positioned at the distal end of the protruding insertion sleeve 24, realizes the insertion part.

FIG. 3e shows a fourth version of the third embodiment of the arrangement according to the present invention. Differently from the version of FIG. 3b, the insertion sleeve 24 as a separate sleeve element is integral with the body part 101″ of the container 1″″. The insertion part is realized by the circumferential section 23 of the stent passage opening 20 embodied at the end of the insertion sleeve 24.

A fifth version of the third embodiment of the arrangement according of the present invention shown in FIG. 3f differs from the fourth version of FIG. 3e in that the separate sleeve element, in the form of an insertion sleeve 24, is not only integral with the body part 101″, but also it extends into an interior of the container 1″″.

In the third embodiment of FIGS. 3a to 3f, the size of the circumference of the stent passage opening corresponds to the size of the diameter of the outer tube 6. Further, the insertion sleeve 24 is of cylindrical shape, wherein the cylindrical diameter corresponds to the size of the diameter of the outer tube 6.

However, in a further embodiment of an arrangement for storage of a self-expanding stent the distal part with the inlet of the insertion sleeve could have a rounded or conical shape creating a funnel passage with a narrowing diameter in direction towards the outer tube 6. In this case the stent passage opening of the container 1 is given by the distal inlet section of the insertion sleeve 24. The retainer 5 is positioned within the inlet section of the insertion sleeve 24 at a diameter that is suitable to keep the stent 3 compressed on the retainer.

In the embodiment of FIGS. 3a to 3f, the first sealing between the stent passage opening of the container 1 and the outer tube 6 can be realized by tight fit of the outer tube 6 within the insertion sleeve 24, because the insertion sleeve 24 is an integral part of the container 1 or of a part thereof. The second sealing 9 is realized as explained for FIGS. 1 and 2.

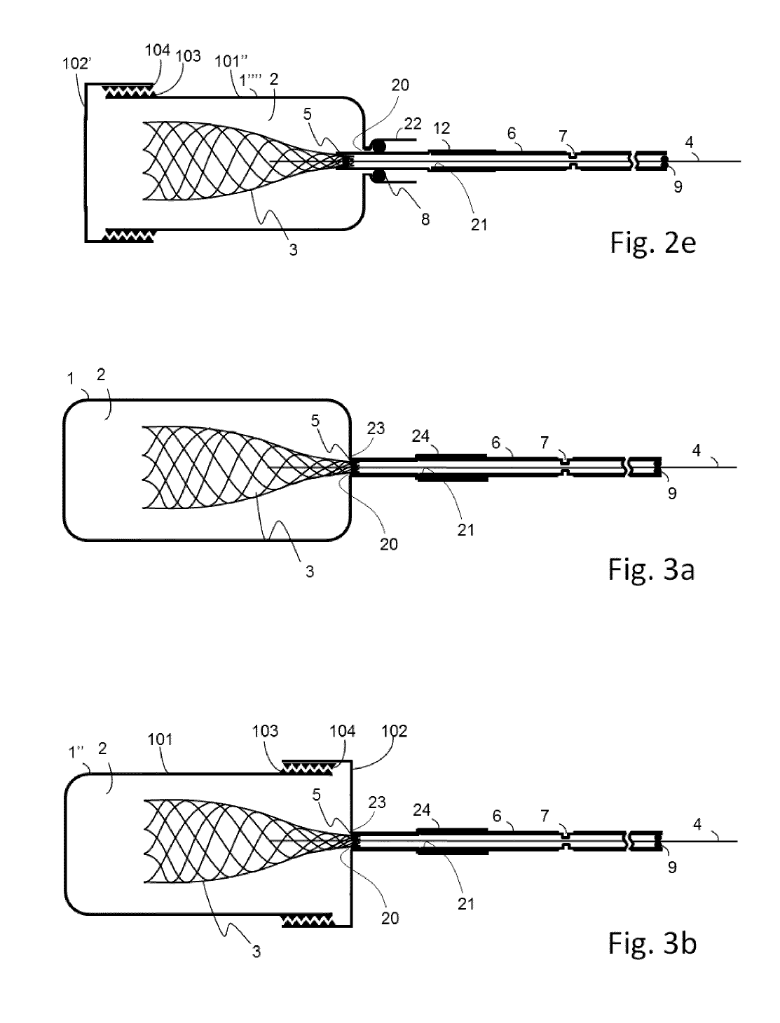

The fifth embodiment as shown in FIGS. 4a to 4c, are combinations of the embodiments of FIGS. 1a to 3f. Again, in FIGS. 4a to 4c, the insertion part is realized by a circumference of the stent passage opening 20 like the circular edge 23 of the stent passage opening 20. For connecting the outer tube 6 to the stent passage opening, the container includes an insertion sleeve 24 as a separate sleeve element similar to the embodiment of FIG. 3. The inner shaft 4 carrying the retainer 5 is axially positioned within the stent passage opening such that the circumference of the stent passage opening is located radially over the retainer 5 and compresses the proximal end of the stent 3 on the retainer 5. The proximal part of the insertion sleeve 24 includes a reception sleeve 22′ analogue to the reception sleeve 22 of the embodiment shown in FIGS. 1a to 2e. A ring-seal is fitted into the circumferential space between the inner surface of the reception sleeve 22′ and the outer surface of the outer tube 6 for sealing off the interior space 2 of the container additionally or instead of the tight fit of the outer tube 6 in the insertion sleeve 24.

According to a method for storing the self-expanding stent 3 in the container 1 or 1′ as shown in FIGS. 1a to 4c, the stent and the components of the delivery system may be cleaned prior to introducing the stent into the container, preferably the stent undergoes a surface treatment for increasing the hydrophilic properties of the stent surface. Then, the proximal end of the stent 3 is compressed onto the retainer 5 and the ring-shaped insertion part is placed over the compressed proximal end of the stent 3 or the retainer 5 is positioned within the ring-shaped insertion part, respectively. Next, the retainer 5 is retracted through the respective insertion part of the embodiments of FIGS. 1a to 4c, i.e., through the distal end of the outer tube 6, the sleeve element 12 or the insertion sleeve 24, into the outer tube 6 or into the separate sleeve element 12, 24 by pulling the inner shaft 4 relative to the outer tube 6 for loading the stent 3 into the outer tube 6 or the separate sleeve element 12, 24, respectively. Then, as defined by one of the embodiments of FIGS. 1a to 4c, the outer tube 6 including the stent 3 is connected to the stent passage opening 20 or to the insertion sleeve 24 or the sleeve element 12, respectively, or the separate sleeve element 12, 24 including the stent 3 is connected to the stent passage opening 20. Then, the stent 3 is introduced into the container 1 by pushing the retainer 5 forward within the outer tube 6 or the separate sleeve element 12, 24, respectively, until only the proximal end of the stent 3 remains compressed by the respective insertion part. Inside the container 1, the majority of the length of the stent 3 remains in an expanded state. The container 1 is filled with an inert filling and the first sealing 8 is put in place, in case the sealing is not already in a tight fit, by pushing the outer tube 6 or the sleeve element 12 through the ring-sealing.

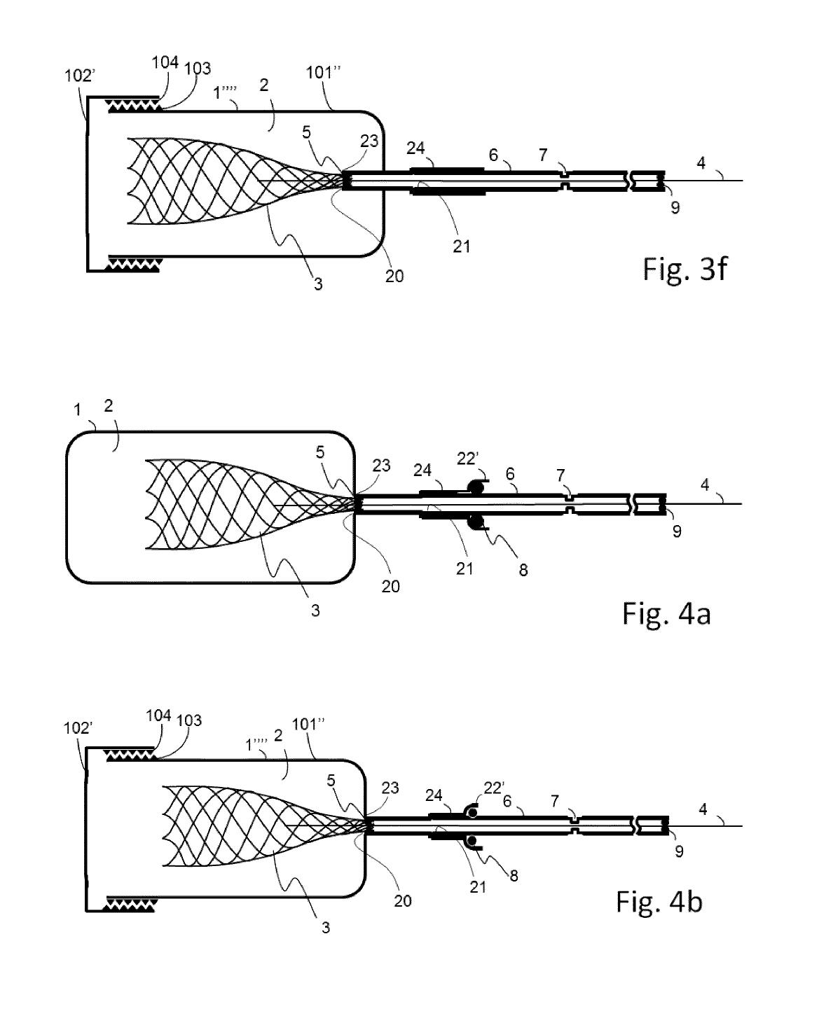

The arrangements of the embodiments shown in FIGS. 5a, 5b, and 6 includes a container 1 with a stent passage opening 20′ much larger than the stent passage opening 20 of the embodiments shown in FIGS. 1a to 4c. The diameter of the stent passage opening 20′ is big enough so that a stent in an expanded state can be introduced into the container. Therefore, the stent can be prepared as explained above. The proximal end of the stent 3 is compressed onto the retainer 5 and the ring-shaped insertion part overlaps the retainer 5. The stent with the compressed proximal end is introduced into the container through the large stent passage opening 20′ while the rest of the stent 3 can be in expanded state. For these embodiments, it is not necessary to load the stent into the outer tube for introducing the stent into the container. The container 1 includes a reception sleeve 22 around the stent passage opening 20′ for accommodating a first sealing 8′ as mentioned before. Because of the large size of the stent passage opening 20′ the ring-seal used for the first sealing 8′ needs to be larger than the ring-seal used for the first sealing 8 for sealing a small stent passage opening 20. In addition, the first sealing 8′ may be a two part sealing.

In FIG. 5a the insertion part is given by a sleeve element 12 as separate sleeve element as explained for the embodiment of FIGS. 2a to 2e. The sleeve element 12 overlaps with the retainer 5 to secure the proximal end of the stent in a compressed state on the retainer as explained above. In FIG. 5b the insertion part is also given by a sleeve element 12′ as separate sleeve element. But the sleeve element 12′ does not radially overlap with the retainer 5′. In this embodiment, the retainer 5′ is designed to engage with the web pattern of the stent also in radial direction to secure the proximal end of the stent in a compressed state on the retainer. The retainer 5′ may for example have cuttings in circumferential direction which grasp the stent. The retainer 5′ may engage with the stent similar to a bayonet fitting. In addition, the stent may have radiopaque markers at it proximal end. Such markers help the user to determine the proximal end of the stent under the x-ray during the application procedure. So the retainer 5′ may be designed in a way that it engages with the markers. The markers or any other parts of the stent structure that are attached to the stent shall be considered as being part of the web pattern. The distal end of the sleeve element 12′ can be slightly modified to simplify the insertion process of feeding the stent into the ring-shape of the sleeve element 12′. For example, the sleeve element 12′ may have an enlarged distal end, e.g., a funnel shaped distal end. Such an arrangement allows for a more flexible positioning of the stent within the interior space.

In FIG. 6 the insertion part is given by the outer tube 6 as explained for the embodiment of FIGS. 1a and 1b.

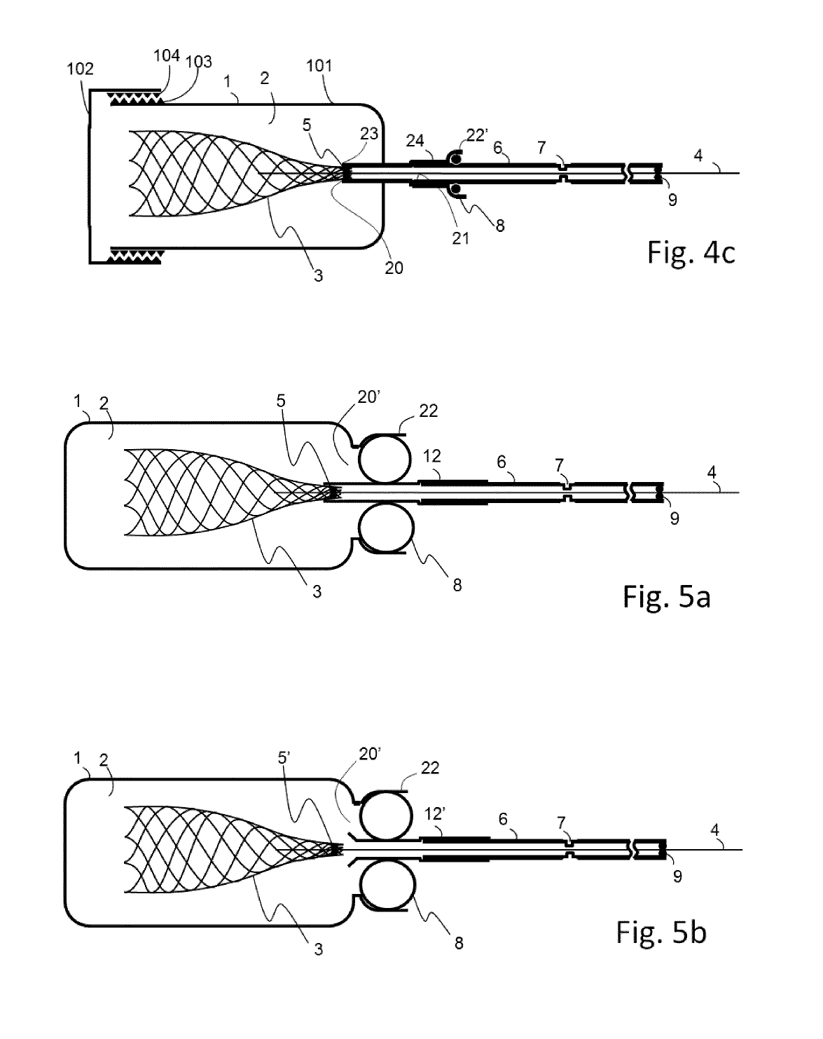

The process of preparing the stent for delivery into a body lumen using the stent delivery system, i.e., of loading the stent into the outer tube 6 and the sleeve element 12 respectively, is basically the same for all the embodiments of FIGS. 1a to 6. The process will be explained by way of example for the embodiment of FIGS. 1a and 1b in combination with FIGS. 7a to 7c. The arrangement according to FIG. 1a or 1b is used as a packaging for the stent 3, wherein the stent is in a storage stage as defined above. Now, when the stent needs to be loaded into the outer tube 6, the retainer 5 is retracted within the outer tube 6 by pulling the inner shaft 4 relative to the outer tube 6 as indicated by arrow 10. As a result, the stent is pulled through the insertion part with an inner diameter that corresponds to the outer diameter of a stent in a compressed and loaded condition. By pulling the stent though the insertion part the stent is fully compressed to the required loading diameter. A stent with a closed cell design, e.g., a braided design, easily slips through the insertion part. A funnel passage as used in the prior art or in FIG. 5b is not compulsory. By further pulling the inner shaft the stent is fully inserted into the outer tube, while the stent remains in the inert filling of the interior space of the container all the time.

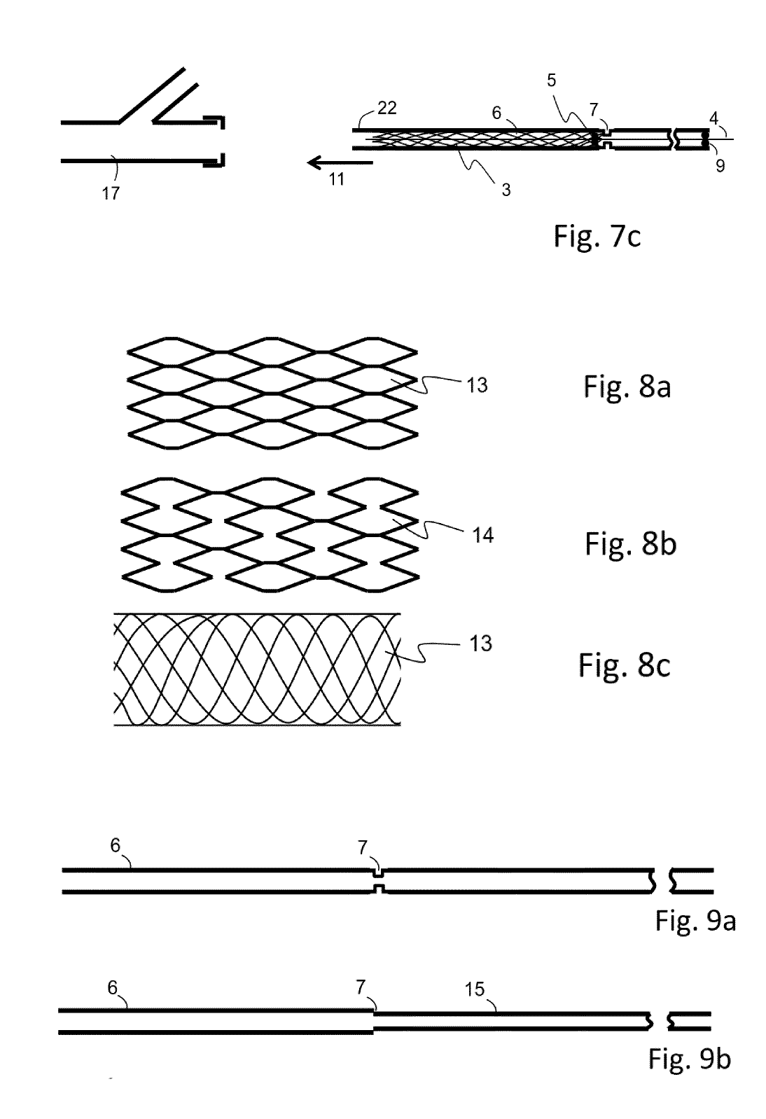

As shown in FIG. 7a the stent is retrieved into the outer tube until the retainer 5 or the stent 3 is blocked by a stopper 7 within the outer tube 6. The stopper 7 is positioned at a distance from the distal end 21 of the outer tube 6 that corresponds to the length of the stent in a fully compressed condition or is at least slightly larger. In the embodiments of FIGS. 1a to 6, the stopper 7 is designed as a radial protrusion extending into the outer tube 6. Other designs for the stopper 7 are described in FIGS. 9a to 9c.

The abutment of the retainer 5 at the stopper 7, when pulling the inner shaft 4, indicates that the stent 3 is in the fully loaded state. Because of the stopper 7 the pulling force applied by the operator is transferred to the outer tube 6, which is pulled out of the stent passage opening 20, through which the outer tube protruded into the interior space 2 of the container 1. Alternatively, and once the stent has been fully retrieved into the outer tube 6, the operator can also pull on the outer tube 6 in proximal direction indicated by arrow 10 to pull out the outer tube 6 from the container 1. In other words, continuous pulling of the inner shaft 4 inserts the stent 3 into the outer tube 6 and detaches the outer tube 6 and the delivery system, respectively, from the container 1, as shown in FIG. 7b. The delivery system with the loaded stent is now prepared for transporting the stent to an application site in the body lumen. As for example shown in FIG. 7c, the outer tube 6 including the loaded stent 3 is inserted in distal direction as indicated by arrow 11 into a Y-connector 17, which is attached to an introducer sheath providing access into the body lumen.

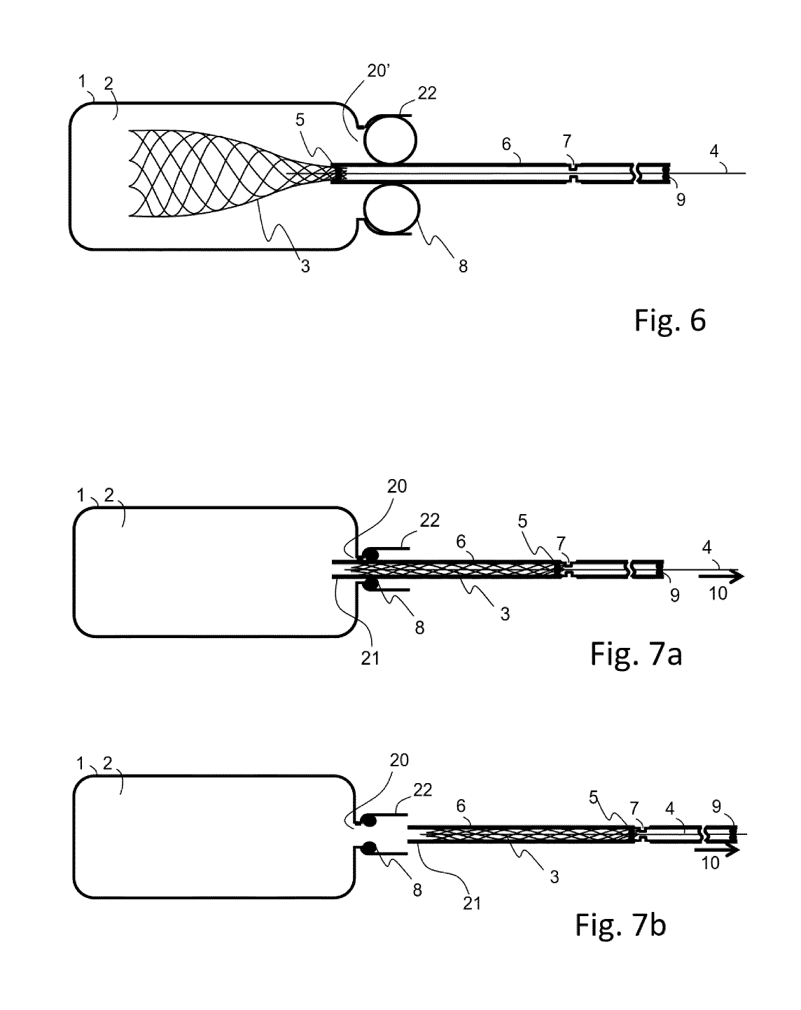

FIGS. 8a, 8b, and 8c show a web pattern of a self-expanding stent as used in the present invention. In general, a stent has a multiplicity of webs that together form a tubular shape. The web-like structure of the stent consists of single cells which form the web. The stent length and, as a passage, the stent lumen with a compressible diameter extend between a proximal and a distal end. The stent assumes an expanded diameter in the dilated or released state, for example for supporting the blood vessel. The stent surface advantageously is embodied in a hydrophilic fashion to promote hemocompatibility.

In order to fully retrieve the stent 3 into the outer tube 6, when using an arrangement according to the invention without a funnel passage, the stent is supposed to have a closed cell design or a braided design as shown in FIGS. 8a and 8c. Closed cell design means that each corner of each cell is connected to a corner of the neighboring cell. A closed cell is indicated with number 13 in FIG. 8a for a stent structure that is produced by a slotted tube or welded wires. Further, FIG. 8c shows a closed cell design of a braided stent.

In contrast, an open cell design means that some corners of cells are not connected to any other cells as indicated by number 14 in FIG. 8b. Open cell stent designs usually have a higher flexibility as compared to closed cell stent designs. On the other hand, closed cell stent designs usually have a higher radial (outward) force as compared to open cell stent designs. Open cell stent design are not suitable to be retrieved into the outer tube 6 without a funnel passage because stent struts of the open cells that are not connected to other struts are likely to be caught at the insertion part during the movement into the proximal direction. For open cell stents an arrangement as described in FIG. 3 could be used, in case the insertion sleeve 24 of container 1 is provided with a rounded edge or a conical section forming a funnel passage leading into the outer tube 6. Also for open cell stents an arrangement as described in FIG. 5b could be used.

FIGS. 9a to 9c illustrate alternative examples of a stopper for blocking an insertion movement of the stent when the retainer is pulled by the inner shaft 4 into the outer tube 6. The stopper can be established by a constriction of the outer tube 6. FIG. 9a corresponds to a stopper 7 as discussed for the embodiments of FIGS. 1a to 6. Another example is shown in FIG. 9b. A stopper 7′ is established by connecting two different tubes together, e.g., by welding, gluing or the like. Each tube has a different inner diameter, whereas the first tube has the diameter of the outer tube 6 and a second tube 15 has a reduced diameter, which would only allow the inner shaft 4 passing through. The tubes would be attached at the determined location of the stopper 7. A further possibility is to establish a stopper 7′ by extruding the outer tube 6 with a distal part of a diameter as required for accommodating the loaded stent and an adjacent part 16 of smaller diameter. A still further example to establish a stopper is shown in FIG. 9c. A second tube which has an outer diameter equal to the inner diameter of the outer tube 6 is inserted into the outer tube 6. And the inner diameter of the smaller tube does not allow the retainer 5 to pass through so that a stopper 7″ is established by the distal end of this smaller tube. A further possibility, as in the example of FIG. 9c, a stopper 7″ is established by extruding the outer tube 6 with a distal part of an inner diameter as required for accommodating the loaded stent and an adjacent part 16 of smaller inner diameter.

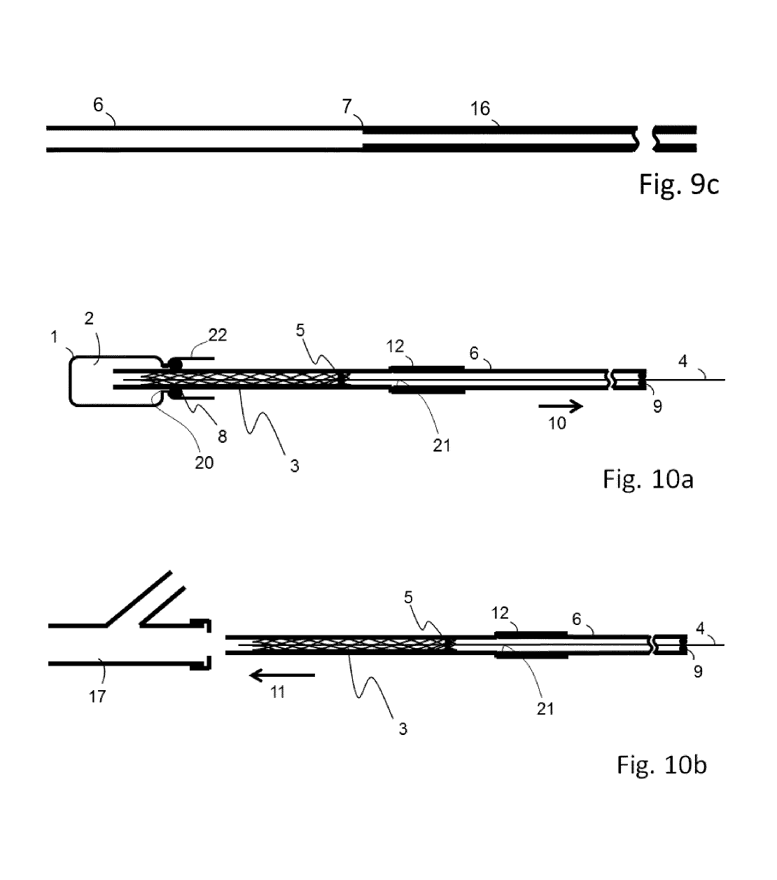

FIGS. 10a and 10b show a method for preparing the stent for delivery into a body lumen as an alternative to the process illustrated in FIGS. 7a to 7c. The method can be used for open and for closed cell stents. In the alternative method, the stent 3 is not pulled back in distal direction 10 all the way into the outer tube 6 as can be seen in FIG. 10a. The stent 3 is accommodated in the sleeve element 12 as a separate element while the sleeve element 12 is detached from the container 1. The container functions as cap or a closure to prevent contaminant particles from penetrating into the sleeve element. The outer tube 6 remains within the proximal end of the sleeve element 12. As shown in FIG. 10b, the sleeve element carrying the compressed stent 3 can be attached to a Y-connector 17 as described for FIG. 7c. Next, the stent 3 can be pushed out of the sleeve element 12 to be implanted at an application site as described before.

The sleeve element 12 can be produced of a more rigid material than the outer tube 6. Thus, it realizes a stiff proximal end of the outer tube 6. In addition, the material of the outer tube 6 could be hardened at the distal end of the outer tube. Then the loading process is accomplished as described for FIGS. 7a to 7c.

FIGS. 11a to 11d show four variants of sealing arrangements for sealing the outer tube 6 and the inner shaft 4 to each other. In particular, such sealing arrangement can be provided at a proximal end of the outer tube 6 of the delivery system of any of the embodiments of an arrangement for storage of a self-expanding stent-like device according to the invention described above. All of these sealing arrangements achieve to limit the volume of interior space 2 and inner volume of outer tube 6, to barrier the inner volumes with regard to contaminations from outside and to prevent leakage of the inert filling out of the interior space 2 through the inner volume of the outer tube 6.

Thereby, FIG. 11a shows, in more detail, the sealing arrangement of the embodiments of the arrangements for storage of a self-expanding stent-like device according to the invention shown in the previous figures.

FIG. 11b shows a second variant of such a sealing arrangement. It includes a second sealing 9 and a shrink tube 91. The ring-shaped second sealing 9 is arranged on or around the inner shaft 4 outside the outer tube 6. The shrink tube 91 is at its one longitudinal end fixedly connected to the outer tube 6 near the proximal end thereof such that the proximal end of the outer tube 6 is arranged inside the shrink tube 91. The shrink tube 91 extends from the outer tube 6 to the second sealing 9 and is fixedly connected to the second sealing 9. The shrink tube 91 is embodied to automatically or elastically reduce its diameter between the proximal end of the outer tube 6 and the second sealing 9.

In FIG. 11c, a third variant of an arrangement for sealing the outer tube 6 and the inner shaft 4 to each other is shown. The sealing arrangement includes a second sealing 9 and a shrink tube 91, which both are essentially identically embodied and arranged as the corresponding elements of the second sealing arrangement shown in FIG. 11b. The third sealing arrangement differs from the second sealing arrangement in that it additionally includes a further second sealing 9′. The further second sealing 9′ is ring-shaped and arranged on or around the outer tube 6 near the proximal end thereof. The shrink tube 91 extends from its portion where it is fixed to the outer tube 6 over the further second sealing 9′ and the proximal end of the outer tube 6 to the second sealing 9 to which it is fixed.

FIG. 11d shows a fourth variant of an arrangement for sealing the outer tube 6 and the inner shaft 4 to each other. It includes a ring-shaped second sealing 9 and a ring-shaped further second sealing 9′ included in a Tuohy Borst adapter 92 having two Tuohy Borst sealings connected to each other. As in the third sealing arrangement shown in FIG. 11c, the second sealing 9 and the further second sealing 9′ are arranged on or around the inner shaft 4 and the outer tube, respectively. The second sealing 9 and the further second sealing 9′ are housed by the Tuohy Borst adapter 92 such that, by screwing portions of the Tuohy Borst adapter 92 relative to each other, the second sealing 9 and the further second sealing 9′ are clamped to or released from the inner shaft 4 or the outer tube 6, respectively.

A rigid distal end of the outer tube or attached to the outer tube allows an easier handling of the loaded stent and also offers a stronger protection of the stent than a rather flexible material can provide for. The use of a stiff distal end at the outer tube of a stent delivery system is not only an advantage when using an arrangement for storage of a self-expanding stent according to the present invention. It is also recommendable for commonly known stent storage or preparation arrangements. Therefore, a stent delivery system including a tube element having a distal end made of a material that is more rigid than the remaining part of the tube element can also be inventive subject matter. Such a stent delivery system is advantageous in particular during preparation of a stent for implantation and also for storage of a stent. Furthermore, the use of such a stiff distal end at the outer tube of a stent delivery system is also useful for open cell self-expanding stent designs.

This description and the accompanying drawings that illustrate aspects and embodiments of the present invention should not be taken as limiting the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the scope of the description and the claims. In some instances, well-known circuits, structures, and techniques have not been shown in detail in order not to obscure the invention. Thus, it will be understood that changes and modifications may be made by those of ordinary skill within the scope of the following claims. In particular, the present invention covers further embodiments with any combination of features from different embodiments described above and below.

The disclosure also covers all further features shown in the figures individually although they may not have been described in the afore. In addition, single alternatives of the embodiments described in the figures and the description and single alternatives of features thereof can be disclaimed from the subject matter of the invention or from disclosed subject matter. The disclosure includes subject matter including the features defined in the claims or the example embodiments, as well as subject matter including these features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. In the context of a given numerate value or range these terms may refer to a value or range that is, e.g., within 20%, within 10%, within 5%, or within 2% of the given value or range. Components described as coupled or connected may be electrically or mechanically directly connected, or they may be indirectly connected via one or more intermediate components. Any reference signs in the claims should not be construed as limiting the scope.

### REFERENCE NUMBERS

1, 1′, 1″, 1′″, 1″″ container
2 interior space
3 stent
4 inner shaft
5, 5′ retainer
6 outer tube
7, 7′, 7″ stopper
8, 8′ first sealing
9, 9′ second sealing
10 arrow proximal direction
11 arrow distal direction
12, 12′ sleeve element as separate sleeve element
13 closed cell of stent
14 open cell of stent
15 second tube
16 adjacent part
17 Y-connector
20, 20′ stent passage opening
21 distal end of outer tube
22, 22′ reception sleeve
23 circular edge
24 insertion sleeve as separate sleeve element
25 further opening
26 cap
91 shrink tube
92 Tuohy Borst adapter
101, 101′, 101″ body part
102, 102′ cover part
103 outer circumferential teeth
104 inner circumferential teeth

1. An arrangement for storage of a self-expanding stent-like device including a container for storing the stent-like device and a stent delivery system arranged at the container and comprising an outer tube and an inner shaft axially moveable relative to the outer tube, wherein:
the container comprises an interior space and one stent passage opening,
a distal end of the outer tube is connected to the stent passage opening,
an insertion part of ring-like shape is located at or near the stent passage opening;
the inner shaft extends through the insertion part and comprises a retainer for preventing the stent-like device at least from axial movement relative to the inner shaft,
wherein, when the stent-like device is stored in the interior space of the container, a proximal end of the stent-like device is compressed to a diameter smaller than a diameter of the insertion part and is held radially compressed by the insertion part or the retainer.

2. The arrangement of claim 1, wherein the insertion part is realized by the distal end of the outer tube or a circumference of the stent passage opening of the container.

3. The arrangement of claim 1, wherein the insertion part is realized by a separate sleeve element arranged on the distal end of the outer tube for connecting the outer tube to the stent passage opening of the container.

4. The arrangement of claim 3, wherein the separate sleeve element is movable relative to the container.

5. The arrangement of claim 3, wherein the separate sleeve element is formed as a portion of the container.

6. The arrangement of claim 1, wherein the container comprises a body part and a cover part mounted to the body part.

7. The arrangement of claim 5, wherein the separate sleeve element is formed as a portion of the cover part of the container.

8. The arrangement of claim 6, wherein the body part of the container comprises an open side which is closed by the cover part.

9. The arrangement of claim 6, wherein the body part of the container comprises a first mount structure and the cover part of the container comprises a second mount structure which is complementary to the first mount structure of the body part.

10. The arrangement of claim 9, wherein the first mount structure of the body part and the second mount structure of the cover part are arranged to fixedly mount the cover part to the body part.

11. The arrangement of claim 9, wherein the first mount structure of the body part and the second mount structure of the cover part are arranged to releasably mount the cover part to the body part.

12. The arrangement of claim 1, wherein the interior space of the container is filled with a storage medium, which is inert in respect of a surface of the stent-like device.

13. The arrangement of claim 1, wherein an inner diameter of the ring-like insertion part corresponds to the outer diameter of the stent-like device in a compressed state.

14. The arrangement of claim 1, wherein a first sealing is provided between the stent passage opening of the container and the insertion part to seal off the interior space of the container.

15. The arrangement of claim 1, wherein the outer tube and the inner shaft are sealed to each other by means of a second sealing.

16. The arrangement of claim 1, wherein a stopper is arranged inside the outer tube at a distance to the distal end of the outer tube, which is at least slightly larger than a length of the stent-like device in a fully compressed state.

17. The arrangement of claim 1, wherein only the proximal end of the stent-like device is compressed and a majority of the stent-like device is in an expanded state.

18. The arrangement of claim 1, wherein the container is made of a rigid material, preferably a transparent material.

19. The arrangement of claim 1, wherein the stent-like device is a stent comprising a closed cell web pattern.

20. The arrangement of claim 19, wherein the stent essentially consists of the closed cell web pattern.

21. The arrangement of claim 1, wherein the stent-like device is a filter device, in particular a vena cava filter.

22. The arrangement of claim 1, wherein an outer profile of the retainer comprises radial recesses for receiving a web pattern of the stent-like device for preventing the stent-like device from axial movement relative to the inner shaft.

23. The arrangement of claim 22, wherein the outer profile of the retainer comprises circumferential cuttings for receiving a web pattern of the stent-like device for preventing the stent-like device from radial movement relative to the inner shaft.

24. The arrangement of claim 1, wherein the insertion part axially extends over the retainer.

25. The arrangement of claim 1, wherein the stent-like device has a hydrophilic surface.

26. A method of storing a self-expanding stent-like device in a container comprising an interior space and one stent passage opening, and preparing the stent-like device for delivery into a body lumen by a stent delivery system comprising an outer tube and an inner shaft axially moveable relative to the outer tube, the method comprising:
compressing at least a proximal end of the stent-like device and compressing the proximal end of the stent-like device onto a retainer provided on the inner shaft,
introducing the stent-like device into the container through the stent passage opening,
storing the stent-like device in the container while the proximal end of the stent-like device is engaged on the retainer and a majority of a length of the stent-like device remains in an at least mostly expanded state,
optionally filling the container with an inert filling for storage of the stent-like device, and
loading the stent-like device into the stent delivery system by retracting the retainer along the outer tube by pulling the inner shaft relative to the outer tube for compressing and inserting the stent-like device via an insertion part of ring-like shape into the outer tube, optionally while the stent-like device is in the inert filling.

27. The method of claim 26, wherein the insertion part is placed over the compressed proximal end of the stent-like device, when the proximal end of the stent-like device is compressed onto the retainer.

28. The method of claim 26, wherein after introducing the stent-like device into the container a first sealing is arranged between the stent passage opening of the container and the insertion part to seal off the interior space of the container.

29. The method of claim 26, wherein after compressing the proximal end of the stent-like device onto the retainer, the retainer is retracted into the outer tube or a separate sleeve element by pulling the inner shaft relative to the outer tube or the separate sleeve element for loading the stent-like device into the outer tube or the separate sleeve element, and introducing the stent-like device into the container by pushing the retainer forward within the outer tube or the separate sleeve element, respectively, until only the proximal end of the stent-like device remains compressed by the retainer or the insertion part placed over the compressed proximal end, while the majority of the length of the stent-like device expands into an at least mostly expanded state within the container.

30. The method of claim 26, wherein after inserting the stent-like device into the outer tube, the outer tube is detached from the container or the separate sleeve element at the container in that the pulling of the inner shaft for loading the stent-like device into the outer tube is continued.
